(19) United States
(12) Patent Application Publication
(10) Pub. No.: US 2004/0266745 A1
(43) Pub. Date: Dec. 30, 2004

(54) Estrogen-gestagen combination preparations and uses thereof

(75) Inventors: Diethard Schwanitz, Eime (DE); Jutta Rogoll, Hannover (DE); Bernd Hachmeister, Isernhagen (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/809,802

(22) Filed: Mar. 26, 2004

Related U.S. Application Data
(63) Continuation of application No. PCT/EP02/10728, filed on Sep. 25, 2002.

(30) Foreign Application Priority Data
Sep. 29, 2001 (EP) 01123511.6

(51) Int. Cl. A61K 31/56

(52) U.S. Cl. 514/182

(57) ABSTRACT

Estragen/gestagen combination preparations useful in hormone replacement therapy to treat, for example, menopausal or climacteric complaints, and method of use thereof.

### CROSS REFERENCE TO RELATED APPLICATIONS

This application is a continuation of international patent application no. PCT/EP02/10728, filed Sep. 25, 2002, designating the United States of America and published in German as WO 03/028735, the entire disclosure of which is incorporated herein by reference. Priority is claimed based on European patent application no. EP 01123511.6, filed Sep. 29, 2001.

### BACKGROUND OF THE INVENTION

The present invention relates to novel estrogen-gestagen combination preparations for use in hormone replacement therapy, e.g., for use in treating menopausal symptoms.

Typical menopausal symptoms occur in conjunction with menopause in women. With the onset of menopause, the ovaries gradually begin to stop functioning so the formation of the female sex hormones estrogen and progesterone declines and ultimately stops entirely. Reproductive ability disappears. The decline in hormone production is associated with numerous changes in the female body which may be manifested in menopausal symptoms, aging of the skin, a decline in bone mass, an increased risk of cardiovascular diseases and a gradual loss of intellectual ability.

To prevent, relieve and/or treat menopausal symptoms, hormone replacement therapy is generally prescribed to act in the body instead of the hormones that are no longer supplied by the ovaries. Hormone preparations, e.g., in the form of pills with an estrogen active ingredient are used in the state of art as monopreparations and as combination preparations with a gestagen as an additional active ingredient. Depending on the patient and the type of combination preparation, the estrogen active ingredient and the gestagen active ingredient may be administered cyclically or continuously, by oral administration of effective dosage units of the hormone active ingredients, usually in regimens without interruption, but optionally also with interruption phases. In a combination preparation currently on the market, the dosage is administered with single and daily doses by oral administration of one pill containing estrogen from days 1 through 14 of the cycle (in women who still have a menstrual cycle) and by oral administration of one pill daily containing a combination of estrogen active ingredient and gestagen active ingredient from days 15 to 28 of the cycle. Another pharmaceutical combination preparation of the state of the art is described in European Patent Application EP 136011 B1. EP 136011 B1 proposes therapeutic combination preparation with low-dose gestagen and estrogen active ingredients for treatment of postmenopausal symptoms in women with an intact uterus; this treatment regimen includes 20 to 120 combined dosage units of gestagen and estrogen plus if desired 3 to 7 separate daily gestagen dosage units (not containing estrogen). These preparations may be administered first by continuous uninterrupted oral administration of gestagen and simultaneous continuous uninterrupted oral administration of estrogen once a day in the form of a single combined dosage unit of the two hormones. On the other hand, estrogen may also be administered cyclically in an oral dose, with the gestagen and estrogen initially being administered for 20 to 120 days once daily in the form of the combined dosage unit and gestagen being administered once a day for 3 to 7 days in the form of the gestagen dosage units.

Menopausal symptoms are often perceived very differently by the individual women affected. The choice of an individual regimen for the prevention, relief and/or treatment of menopausal symptoms therefore makes a flexible possibility for variation of treatment seem desirable, i.e., a treatment regimen that would optionally also allow alternative use regimen with one and the same preparation.

### SUMMARY OF THE INVENTION

Accordingly, it is an object of the present invention to provide an improved estrogen-gestagen formulation.

Another object of the invention is to provide an estrogen-gestagen formulation which facilitates flexible variation of hormonal treatment.

A further object of the invention is to provide an estrogen-gestagen formulation which enables administration of alternative treatment regimens.

Accordingly, the present invention proposes a novel estrogen-gestagen combination preparation with an alternating regimen. The invention also relates to the use of an estrogen and a gestagen for producing an oral pharmaceutical preparation which contains separate daily dosage units (14 or multiple thereof) of an estrogen active ingredient and a corresponding number of a combination of an estrogen active ingredient with an gestagen active ingredient in combined daily dosage units, for the prevention, relief and/or treatment of menopausal symptoms in women, in particular in women with an intact uterus, if a menstrual cycle is no longer desired, by daily alternating uninterrupted administration of the daily dosage units of the estrogen active ingredient and to combined daily dosage units of the combination of the estrogen active ingredient with the gestagen active ingredient over a period of at least 28 days (28-day cycle).

The pharmaceutical preparation according to the invention is suitable for the prevention, relief and/or treatment of menopausal symptoms including pre-, peri- and postmenopausal symptoms in women with an intact uterus when a menstrual cycle is no longer desired. Menopausal symptoms here include a number of adverse effects or pathological changes because of the changing hormone balance. Perimenopausal women (e.g., approximately those more than 40 years of age), menopausal and postmenopausal women often suffer a variety of different states and symptoms which are to be attributed to withdrawal from estrogen because of subsiding function of the ovaries. Such symptoms may vary greatly in duration and they may be manifested, for example, in hot flashes of varying extent, depending on the individual, and in vaginal dryness with an increased susceptibility to infections. The bone mineral loss in particular is associated with a decline in bone mass (osteoporosis) as a long-term menopausal, perimenopausal and postmenopausal condition that is very threatening to a woman's health. In addition, an increased risk of heart attacks and ischemic heart disease due to definitely elevated serum cholesterol levels has also been discussed in the professional medical world. In some women, other symptoms such as depression, insomnia, nervousness or even such conditions as arthritis, etc., may occur in addition to the main symptoms and pathological states mentioned above.

In treatment of menopausal, perimenopausal and postmenopausal symptoms, it is acknowledged that estrogen is the most effective agent for controlling hot flashes and vaginal atrophy and it prevents or retards the occurrence of clinical manifestations of osteoporosis. From a clinical standpoint it is not necessary to administer estrogens alone but instead they are preferably administered in combination with a gestagen in a suitable dosage and/or treatment regimen. The present invention here makes available a combination and type of administration not previously known in the related art for this purpose.

The indications according to the present invention include in particular premenopausal and menopausal symptoms with menstrual disorders, menstrual irregularities (menstrual anomalies), lack of menstrual bleeding (amenorrhea), too little menstrual bleeding (hypomenorrhea), too infrequent menstrual bleeding (oligomenorrhea), excessive menstrual bleeding (hypermenorrhea), spotting between periods and inadequate functioning of the ovaries (ovarian insufficiency).

The preparation according to this invention is administered in an alternating treatment regimen with individual doses and daily doses. This alternating therapy regimen is suitable in particular for the group of patients who no longer wish to have a menstrual cycle. The gestagen is administered here in alternation, as can be seen from FIG. 1 in an exemplary embodiment of this invention, for example. For example if the estrogen active ingredient is formulated in white pills and the estrogen-gestagen combination is formulated in pink pills, then the alternating administration of gestagen is done by administering one white and one pink pill in alternation each day. In other words, on the first day a white pill (1) is taken, on the second day a pink pill (2) is taken, on the third day a white pill (3) is taken again and on the fourth day a pink pill (4) is taken again, etc., until reaching the end of a 28-day cycle.

With this alternating dosage regimen (pink-white regimen), the pills are to be taken in the order of the numbers given, beginning with the number (1) according to a treatment regimen which is printed on the front of a blister pack strip, for example, as shown in FIG. 1. These numbers do not indicate the date but instead indicate the sequence in which the particular pills are to be taken. Other example of embodiments for the arrangement and labeling of the particular pills in inventive forms of administration for the alternating treatment regimen are depicted in FIG. 3 and FIG. 4. Inventive packaged pharmaceutical preparations suitable for this purpose are described in greater detail below.

If the patient is having regular menstrual periods before the beginning of the treatment or when changing to this dosage regimen, the first dose of the medication is begun on day one of menstruation. Women who no longer have a uterus or who are no longer having menstrual periods can begin taking the medication at any time.

After consuming the entire blister pack, i.e., after completing a 28-day cycle, the next pill is taken on the following day, i.e., without a pause, starting with a new blister pack according to the same alternating pink-white regimen as that described above, following the numbers used for orientation purposes.

To facilitate use of the medication, the pills may also be presorted in a pill dispenser, always alternating pink and white for one month (28-day cycle). To explain this aspect of the invention, FIG. 5A and FIG. 5B give an example of the geometric design of a pill dispenser or pill dispenser, i.e., a pill dispenser having a round geometry; other geometric designs, e.g., oval shapes or rectangular shapes are also possible, e.g., by analogy with the blister packs. In addition, this aspect of the invention is described in greater detail below in conjunction with the pharmaceutical preparations packaged according to this invention.

In a preferred variant of this invention, the pharmaceutical preparation includes 14 daily dosage units of an estrogen active ingredient and 14 daily dosage units of a combination of the estrogen active ingredient with a gestagen active ingredient. This pharmaceutical preparation consisting of 14 daily dosage units of each is intended for a one-month treatment, i.e., a 28-day cycle and is packaged accordingly. If desired, the inventive pharmaceutical preparation may also be intended for three months of treatment, i.e., for three successive 28-day cycles and packaged accordingly, i.e., the three times the number of daily dosage units required for a 28-day cycle is made available, each dosage unit containing the estrogen active ingredient on the one hand and the combination of the estrogen active ingredient with a gestagen active ingredient on the other hand. In this three-month variant, the pharmaceutical preparation then includes, for example, three separate package subunits of 14 daily dosage units of an estrogen active ingredient and 14 daily dosage units of a combination of the estrogen active ingredient with a gestagen active ingredient or, for example, three times 14 daily dosage units of an estrogen active ingredient and three times 14 daily dosage units of a combination of the estrogen active ingredient with a gestagen active ingredient. As an alternative, 42 daily dosage units of an estrogen active ingredient and 42 daily dosage units of a combination of the estrogen active ingredient with a gestagen active ingredient may also be provided for the three-month variant.

Estrogens that can be used within the scope of this invention include estrogen active ingredients selected from the group consisting of estradiol, 17β-estradiol, estradiol valerate, natural conjugated estrogens including those of equine origin, estrone, estropipate (piperazine estrone sulfate), ethinyl estradiol, mestranol and quinestranol. Natural conjugated equine estrogens (CEE=conjugated equine estrogens) are preferred. Natural conjugated estrogen hormones are an amorphous extract representing mixtures of water-soluble conjugated estrogens usually obtained from natural sources such as in particular the urine of gravid mares. The main estrogenic component in this mixture is the sodium salt of estrone sulfate and, in a lesser amount, the sodium salt of equiline sulfate. The total estrogenic efficacy of pharmaceutical preparations is usually expressed in the form of equivalent amounts of the sodium estrone sulfate salt and optionally the sodium equiline sulfate salt. Other conjugated estrogenic active ingredient components present in the mixtures of natural conjugated estrogens are also present as water-soluble sodium sulfate salts in the mixtures including for example 17α-dihydroquiline, 17α-estradiol and 17β-dihydroequiline. Tablets for oral administration contain mixtures of the natural conjugated estrogens, usually in amounts of approximately 0.3 mg, 0.625 mg, 0.9 mg, 1.25 mg and 2.5 mg of the conjugated estrogens.

Gestagens that can be used within the scope of this invention include gestagen active ingredients selected from the group consisting of levonorgestrel, dl-norgestrel, norethindrone (norethistrone), norethindrone (norethistrone) acetate, ethynediol diacetate, dydrogesterone, medroxyprogesterone acetate, norethynodrel, allylestrenol, lynoestrenol, quingestanol acetate, medrogestone, norgestrienone, dimethisterone, ethisterone, cyproterone acetate, chlormadinone acetate, megestrole acetate, gestoden, desogestrel, trimegestone, dienogest, drosperinone and nomegestrole acetate. Gestagens that are preferred according to this invention include dydrogesterone, medroxyprogesterone acetate, norethistrone acetate, trimegestone, dienogest, drosperinone and in particular medrogestone.

In one embodiment of this invention, the daily dosage unit of estrogen contains the estrogen active ingredient, preferably a mixture of natural conjugated equine estrogens in an amount of 0.05 mg to 10 mg depending on the type of estrone used, and the combined daily dosage unit contains the estrogen active ingredient, preferably also a mixture of natural conjugated equine estrogens, in an amount of 0.05 mg to 10 mg, depending on the type of estrone used, and contains the gestagen active ingredient in an amount of 0.05 mg to 50 mg, depending on the type of gestagen used. Medrogestone, which is the preferred gestagen for use here, is present in an amount of 1 mg to 20 mg, in particular in an amount of 1 mg to 10 mg. Those skilled in the art will be familiar with the quantities to be used for the particular individual estrogen and/or gestagen active ingredients.

In an especially preferred variant of this invention, the daily dosage unit of the estrogen active ingredient contains 14 mg of an extract from the urine of gravid mares standardized to 0.33 mg sodium salt of estrone 3-hydrogen sulfate and 0.17 mg sodium salt of equiline 3-hydrogen sulfate (corresponding to 0.6 mg conjugated equine estrogens) and the combined daily dosage unit consists of a combination of 14 mg of an extract from the urine of gravid mares standardized to 0.33 mg sodium salt of estrone 3-hydrogen sulfate and 0.17 mg sodium salt of equiline 3-hydrogen sulfate (corresponding to 0.6 mg conjugated estrogens) as the estrogen active ingredient and another 5 mg medrogestone as the gestagen active ingredient.

The active ingredients are in the daily dosage units where they are mixed in a suitable manner with the conventional pharmaceutical vehicles and excipients, for example, as an orally administerable solid galenical formulation, e.g., as tablets, pills or coated pills in any geometric design in particular. To produce these solid oral pharmaceutical forms, the usual galenical preparation methods may be used, including in addition to tableting or pill production methods, if desired, film coating methods and pill coating methods. Those skilled in the art will be familiar with these methods. For example the pharmaceutical materials known in the related art may be considered for the pharmaceutical vehicles and excipients such as the usual organic and/or inorganic vehicle substances such as lactose, talc or starch, for example, and other excipients that are conventionally used pharmaceutically as needed such as matrix forming substances, binders, granulation aids, lubricants, slip agents, humectants or desiccants, fillers, coloring agents or tablet disintegrants. In addition other excipients may also be added such as preservatives, stabilizers, taste correcting agents, flavoring agents and the like. Sheathed solid forms of administration may be in the form of film tablets (film or varnish tablet), optionally with an additional pill coating layer or as traditional coated pills. Film tablets here are usually sheathed with a polymer film. Formulations for sheathing film tablets include for example plasticizers, dispersion aids or smoothing aids, covering agents and coloring agents, for example, in addition to the polymer film-forming agent; the ingredients are usually combined with a solvent, usually ethanol or acetone, for application to the tablet core or pill core. Coated pills are usually the solid form of administration sheathed with a sugar. Pill coating syrup, i.e., highly concentrated sugar solutions with a sugar content of up to 80 wt % are generally used for pill coating. Sucrose is the main sugar used here. Different layers are applied one after the other and in portions to a core in the essentially known pill coating methods, e.g., the cover layer of a few portions of syrup coating and powder coating dispersed in between; the application layer of multiple portions of application syrup and application powder dispersed in between; a smoothing layer of pure syrup, possibly colored, to impart a brilliance to the pill surface; followed by glazing or polishing with a wax or a wax solution as the final step.

Examples of vehicles and excipients that can be used include inert inorganic materials such as tribasic calcium phosphate, calcium sulfates or titanium dioxide or inert organic materials such as carnauba wax, cellulose, glycerol monooleate, lactose, magnesium stearate, methyl cellulose, pharmaceutical glaze, polyethylene glycol, stearic acid, sucrose. Preferred oral pharmaceutical preparations according to the present invention include for example materials such as gum arabic, carmellose sodium, carnauba wax, lactose, Macrogol 4000 and 6000, magnesium oxide, magnesium stearate, cornstarch, povidone, sucrose, shellac, talc, bleached wax, coloring agents E104, E127 and E171.

The solid oral daily dosage units may also be pigmented, if desired, e.g., for identification and differentiation of the estrogen dosage units and combined estrogen-gestagen dosage units. As an alternative or in addition, the identification and differentiation may, if desired, also be accomplished via the packaging material in which the daily dosage units with the estrogen active ingredient on the one hand and with the combined estrogen-gestagen active ingredients on the other hand are made available. The dosage units may be identified on the basis of color and/or number, for example.

The invention additionally relates to packaged pharmaceutical preparations (package units) which comprise

a packaging material which separately accommodates 14 daily dosage units (or an integral multiple thereof) of an estrogen active ingredient and a corresponding number of a combination of an estrogen active ingredient with a gestagen active ingredient in combined daily dosage units, and the daily doses of the estrogen active ingredient on the one hand and the combined daily doses of the estrogen active ingredient and the gestagen active ingredient on the other hand are marked in a suitable manner to clearly differentiate them from one another, and

a label and/or a package insert to indicate that the preparation may be administered for the prevention, relief and/or treatment of menopausal symptoms in women, especially in women with an intact uterus when a menstrual cycle is no longer desirable, by uninterrupted daily administration of the daily dosage units, alternating each day, of the estrogen active ingredient and the combined daily dosage units of the combination of the estrogen active ingredient with the gestagen active ingredient over a period of at least 28 days (28-day cycle).

In a preferred embodiment, the packaged pharmaceutical preparation according to the invention is characterized in that the daily dosage unit with the estrogen active ingredient on the one hand and the daily combined dosage unit of the estrogen active ingredient and the gestagen active ingredient on the other hand are identified by visual marking, preferably by a difference in color to differentiate them from one another.

In an especially preferred embodiment, the inventive packaged pharmaceutical preparation includes as the packaging material a blister pack on which is printed a system for facilitating the alternating administration of the daily dosage units of the estrogen active ingredient and the combined daily dosage unit of estrogen active ingredient and gestagen active ingredient, preferably as a scheme using integers from 1 to 28 to record the sequence of the particular dosage units to be administered each day.

To facilitate daily usage of the particular appropriate dosage unit, they may be presorted alternately in a pill dispenser that is subdivided into compartments, e.g., for a month (28 days) or they may be presorted subsequently from a blister pack. This invention therefore also concerns packaged pharmaceutical preparations as the package units which comprise

a packaging material which separately accommodates. 14 daily dosage units (or an integral multiple thereof) of an estrogen active ingredient and the corresponding number of a combination of an estrogen active ingredient with a gestagen active ingredient in combined daily dosage units, and the daily doses of the estrogen active ingredient on the one hand and the combined daily doses of the estrogen active ingredient and the gestagen active ingredient on the other hand are labeled in a suitable manner to clearly differentiate one from the other, and

a label and/or a package insert to indicate that the preparation may be administered for the prevention, relief and/or treatment of menopausal symptoms in women, especially in women with an intact uterus when a menstrual cycle is no longer desirable, by uninterrupted daily administration of the daily dosage units, alternating each day, of the estrogen active ingredient and the combined daily dosage units of the combination of the estrogen active ingredient with the gestagen active ingredient over a period of at least 28 days.

In a preferred embodiment, the inventive packaged pharmaceutical preparation described above is characterized in that the packaging material is a pill dispenser subdivided into 28 compartments, preferably a pill dispenser subdivided into 28 compartments with these compartments arranged in a ring accommodating a daily dosage unit of an estrogen active ingredient which alternates with a combined daily dosage unit of an estrogen active ingredient and a gestagen active ingredient, with the dosage units labeled appropriately so that they are clearly differentiable.

If desired, the individual compartments may be labeled by the days of the week, thereby supplementing the color labeling of the dosage units.

Blister packs may be advantageously used as the packaging material for distribution of the pharmaceutical preparation and the pill dispensers described above are only distributed along with the blister pack as a supportive measure.

As an alternative to the alternating treatment regimen described for the inventive preparations, the gestagen may also be administered according to a traditional cyclic treatment regimen which may if desired also be printed on a front side of the blister pack for example in addition to the alternating treatment regimen as an option for selection given on the back side of the blister pack. The packaged pharmaceutical preparations may thus provide two different treatment regimens, e.g., on a blister pack and the user may select between the two dosage regimens provided, whereby the treating physician decides at the start of treatment which of the two regimens is suitable for the patient in question. The cyclic treatment regimen which is additionally provided, optionally as a selection option is suitable in particular for female patients who still are having regular menstrual periods and would like to continue to do so. The gestagen is administered cyclically here, e.g., as shown in an illustrative embodiment in FIG. 2; other embodiments are also conceivable. For example if the estrogen active ingredient is formulated in white pills and the estrogen-gestagen active ingredient combination is formulated in pink pills, then the alternating administration of gestagen is accomplished by administered one white pill daily from day 1 through day 14 of the cycle and one pink pill daily from day 15 through day 28 of the cycle.

In this cyclic dosage regimen, the pills are taken according to a treatment regimen printed on the front of a blister pack strip, beginning at the starting point and continuing in the order of the arrows, as illustrated in the embodiment shown as an example in FIG. 2, for example. In the case of inventive embodiments of a blister pack according to FIG. 3 or FIG. 4, for example, the selection option of the traditional cyclic treatment regimen may be printed on the front side of the blister pack, e.g., according to the arrangement of the particular pills and likewise indicated by arrows or numbers.

After the pills in the blister pack have been used up, i.e., after conclusion of a 28-day cycle, administration is resumed with a new blister pack according to the same cyclic treatment regimen on the following day, i.e., without a pause in usage, as described above, following the arrows that are used for orientation.

If the patient has still been having regular monthly periods before the start of the treatment, the first dose is taken on day 1 of her menstrual cycle or when changing to the dosage regimen. Women who no longer have a uterus or who are no longer having a menstrual cycle can begin taking the medication at any time.

A special advantage of this invention, in particular the packaged pharmaceutical preparations, is that patient compliance with the treatment is greatly increased due to the form of administration. This is true in particular of the packaged pharmaceutical preparations which are designed so that they also allow a traditional cyclic treatment regimen in addition to the alternating treatment according to this invention as a selection option because it is possible to avoid a change in preparations when switching from one treatment regimen to the other. Therefore, the patients affected by such a switch can continue to take the preparation in the usual formulation which has proven to be tolerated individually well and with a switch it is possible to prevent from the beginning any intolerance which might otherwise occur.

### BRIEF DESCRIPTION OF THE DRAWINGS

The invention is described in further detail with reference to illustrative embodiments depicted in the accompanying drawing figures in which:

FIG. 1 shows the back side of a blister pack according to this invention with an imprint of the dosage regimen expressed in the form of a numerical scheme;

FIG. 2 shows the front side of the blister pack with an alternative dosage regimen expressed in the form of an arrow scheme for cases in which menstrual bleeding is still desired;

FIG. 3 shows the back side of a blister pack according to this invention with a printed dosage regimen in the form of a numerical scheme;

FIG. 4 shows the front side of the blister pack with a printed dosage regimen according to the invention in the form of an arrow scheme, and

FIGS. 5A and 5B, respectively, show a pill dispenser without contents and a pill dispenser with contents.

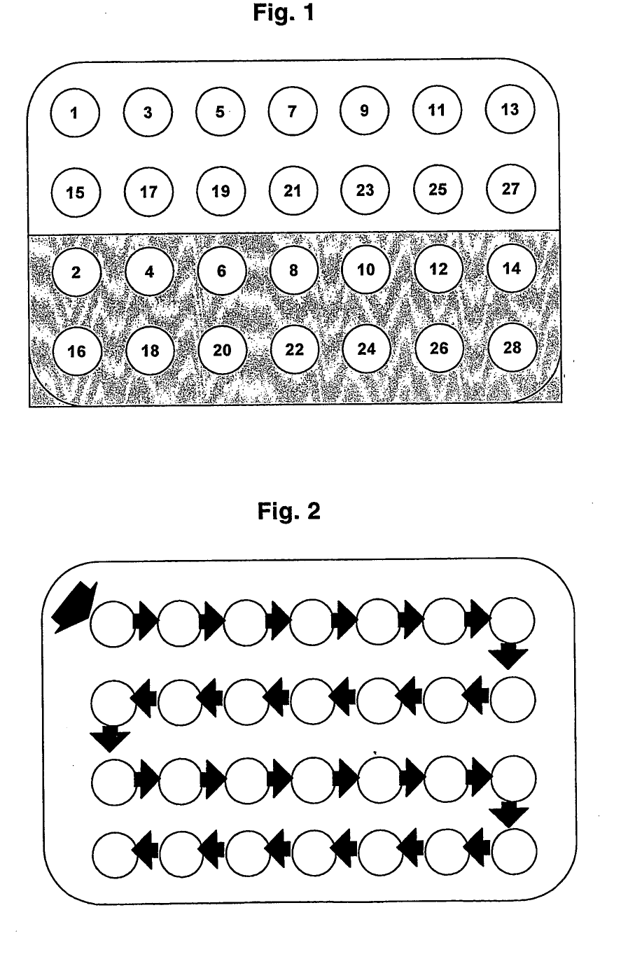

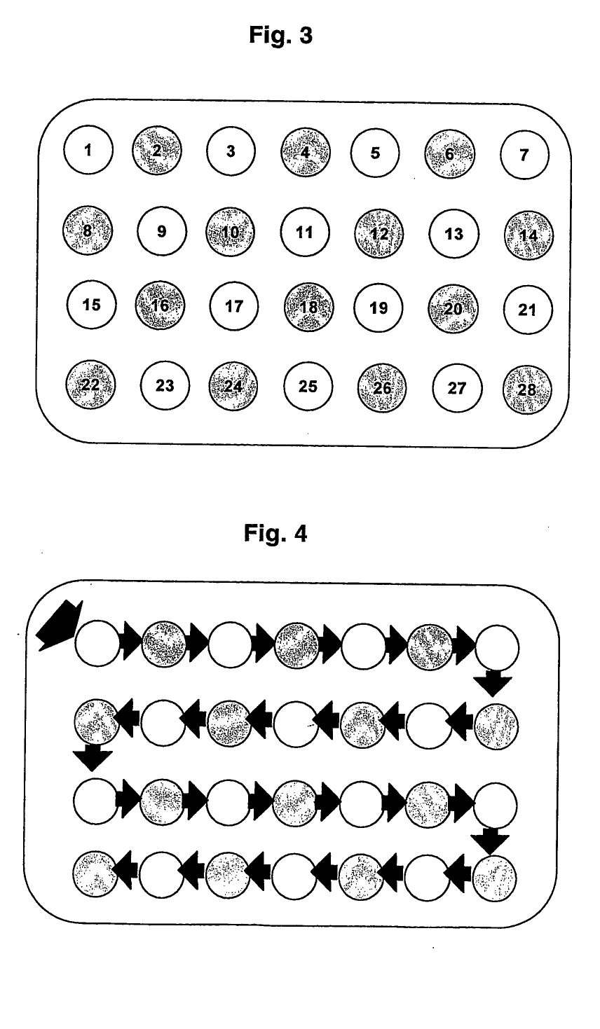

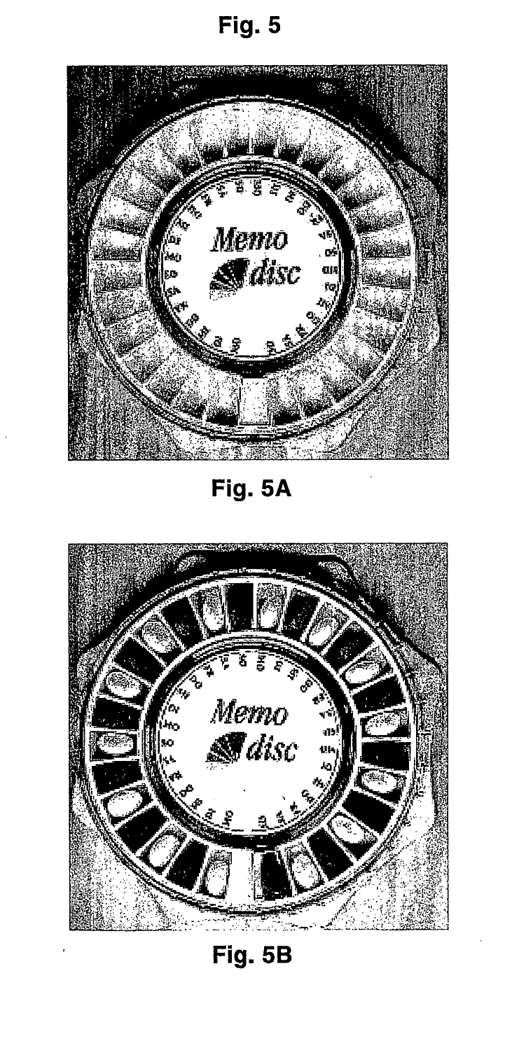

### EXAMPLE

The following example is presented to illustrate this invention through an illustrative concrete pharmaceutical preparation, without limiting the scope of this invention.

### Example

An example of a package unit (packaged pharmaceutical preparation) contains the combination preparation consisting of the two different hormone pills, namely white pills containing an estrogen active ingredient and pink pills containing an estrogen and a gestagen. The estrogen content is obtained as a standardized extract from the urine of gravid mares. The gestagen component (corpus luteum hormone content) consists of medrogestone. One package unit contains 14 white pills and 14 pink pills.

One white pill contains as the active pharmaceutical ingredients 14 mg of an extract from the urine of gravid mares, standardized to 0.33 mg sodium salt of estrone 3-hydrogen sulfate and 0.17 mg sodium salt of equiline 3-hydrogen sulfate (corresponding to 0.6 mg conjugated equine estrogens).

One pink pill contains as the active pharmaceutical ingredients 14 mg of an extract from the urine of gravid mares, standardized to 0.33 mg sodium salt of estrone 3-hydrogen sulfate and 0.17 mg sodium salt of equiline 3-hydrogen sulfate (corresponding to 0.6 mg conjugated estrogens) and 5 mg medrogestone.

Other ingredients in both pills (white and pink): gum arabic, carmellose sodium, carnauba wax, lactose, Macrogol 4000 and 6000, magnesium oxide, magnesium stearate, cornstarch, povidone, sucrose, shellac, talc, bleached wax, coloring agents E104, E127 and E171.

Packaged pharmaceutical preparations may have the following form of administration and content, for example:

(a) Pills in a package containing a total of 28 pills (one-month package for a 28-day cycle; 14 white pills and 14 pink pills); or

(b) Pills in a package containing a total of 84 pills (quarter-year package with 3×28 pills for three 28-day cycles; 3×14 white pills and 3×14 pink pills).

The foregoing description and example have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of treating or inhibiting menopausal symptoms in a woman when a menstrual cycle is no longer desired, said method comprising orally administering to said woman over a period of at least 28 days uninterrupted alternating daily dosage units of an estrogen active ingredient and combined daily dosage units of a combination of estrogen active ingredient and a gestagen active ingredient.

2. A method according to claim 1, wherein said woman is a woman with an intact uterus.

3. An orally administrable pharmaceutical preparation for treating or inhibiting menopausal symptoms in a woman when a menstrual cycle is no longer desired, said pharmaceutical preparation containing 14 separate daily dosage units or an integral multiple thereof of an estrogen active ingredient and a corresponding number of combined daily dosage units of a combination of estrogen active ingredient with a gestagen active ingredient, said daily dosage units facilitating daily alternating uninterrupted administration of daily dosage units of the estrogen active ingredient and the combined daily dosage units of the combination of estrogen active ingredient and gestagen active ingredient over a period of at least 28 days.

4. An orally administrable pharmaceutical preparation according to claim 3, wherein said pharmaceutical preparation contains 14 daily dosage units of an estrogen active ingredient and 14 combined daily dosage units of a combination of estrogen active ingredient and a gestagen active ingredient for a 28-day cycle.

5. An orally administrable pharmaceutical preparation according to claim 3, wherein said pharmaceutical preparation contains 42 daily dosage units of the estrogen active ingredient and a corresponding number of combination daily dosage units containing a combination of estrogen active ingredient with a gestagen active ingredient, whereby alternating daily doses of said estrogen active ingredient and daily combination doses of a combination of estrogen active ingredient and a gestagen active ingredient can be administered over a 12 week period of time.

6. An orally administrable pharmaceutical preparation according to claim 3, wherein said estrogen active ingredient is selected from the group consisting of estradiol, 17β-estradiol, estradiol valerate, natural conjugated estrogens including those of equine origin, estrone, estropipate (piperazine estrone sulfate), ethinyl estradiol, mestranol and quinestranol.

7. An orally administrable pharmaceutical preparation according to claim 6, wherein said estrogen active ingredient is natural conjugated equine estrogens.

8. An orally administrable pharmaceutical preparation according to claim 3, wherein said gestagen active ingredient is selected from the group consisting of levonorgestrel, dl-norgestrel, norethindrone (norethistrone), norethindrone (norethistrone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, cyproterone acetate, chlormadinone acetate, megestrole acetate, gestoden, desogestrel, trimegestone, dienogest, drosperinone and nomegestrole acetate.

9. An orally administrable pharmaceutical preparation according to claim 8, wherein said gestagen active ingredient is selected from the group consisting of dydrogesterone, medroxyprogesterone acetate, norethistrone acetate, trimegestone, dienogest, drosperinone and medrogestone.

10. An orally administrable pharmaceutical preparation according to claim 9, wherein said gestagen active agent is medrogestone.

11. An orally administrable pharmaceutical preparation according to claim 3, wherein the daily dosage units of estrogen active ingredient contain a mixture of natural conjugated equine estrogens in an amount of 0.05 mg to 10 mg, and the combined daily dosage units contain the estrogen active ingredient in an amount of 0.05 mg to 10 mg, and the gestagen active ingredient in an amount of 0.05 mg to 50 mg.

12. An orally administrable pharmaceutical preparation according to claim 11, wherein the combined daily dosage units contain a mixture of natural conjugated equine estrogens as the estrogen active ingredient and medrogestone in an amount of 1 mg to 20 mg as the gestagen active ingredient.

13. An orally administrable pharmaceutical preparation according to claim 12, wherein the combined daily dosage units contain an amount of 1 mg to 10 mg or medrogestone as the gestagen active ingredient.

14. An orally administrable pharmaceutical preparation according to claim 3, wherein the daily dosage units of the estrogen active ingredient contain about 14 mg of an extract from the urine of gravid mares, standardized to 0.33 mg sodium salt of estrone 3-hydrogen sulfate and 0.17 mg sodium salt of equiline 3-hydrogen sulfate (corresponding to 0.6 mg conjugated equine estrogens), and the combined daily dosage units contain a combination of about 14 mg of an extract from the urine of gravid mares, standardized to 0.33 mg sodium salt of estrone 3-hydrogen sulfate and 0.17 mg sodium salt of equiline 3-hydrogen sulfate (corresponding to 0.6 mg conjugated estrogens) as the estrogen active ingredient and about 5 mg medrogestone as the gestagen active ingredient.

15. A packaged pharmaceutical preparation for hormone replacement therapy containing:
a packaging material in which 14 daily dosage units or an integral multiple thereof of an estrogen active ingredient and a corresponding number of combined daily dosage units containing a combination of an estrogen active ingredient with a gestagen active ingredient are accommodated separately, and the daily doses of the estrogen active ingredient and the combined daily dosage units of the estrogen active ingredient and the gestagen active ingredient are labeled or characterized to differentiate them from one another, and
a label or package insert indicating that the preparation may be administered for treating or inhibiting menopausal symptoms in women when a menstrual cycle is no longer desired, by uninterrupted alternating daily administration of the daily dosage units of the estrogen active ingredient and the combined daily dosage units of the combination of estrogen active ingredient and gestagen active ingredient over a period of at least 28 days.

16. A packaged pharmaceutical preparation according to claim 15, wherein the daily dosage unit containing the estrogen active ingredient and the daily combined daily dosage unit of estrogen active ingredient and gestagen active ingredient are visibly labeled by different colors for differentiation from one another.

17. A packaged pharmaceutical preparation according to claim 15, wherein the packaging material is a blister pack on which is printed a schedule to facilitate alternating administration of the daily dosage units of the estrogen active ingredient and the combined daily dosage unit of the estrogen active ingredient and gestagen active ingredient.

18. A packaged pharmaceutical preparation according to claim 17, wherein said schedule is printed out as a diagram of integers from 1 to 28.

19. A packaged pharmaceutical preparation according to claim 17, wherein in addition to the schedule for alternating administration of the daily dosage units of estrogen active ingredient and combined daily dosage unit of estrogen active ingredient and gestagen active ingredient, the blister pack additionally has a second cyclic schedule provided on an opposite side specifying initial administration of 14 consecutive daily dosage units of the estrogen active ingredient followed by 14 combined daily dosage units of estrogen active ingredient and gestagen active ingredient for women in whom a menstrual cycle is still desired.

20. A packaged pharmaceutical preparation according to claim 15, wherein the packaging material comprises a pill dispenser subdivided into 28 compartments in which daily dosage units of estrogen active ingredient and combination daily dosage units of estrogen active ingredient and gestagen active ingredient are accommodated in alternation and are labeled to differentiate them from one another.

21. A packaged pharmaceutical preparation according to claim 19, wherein said compartments are arranged in ring shape.
